(19) United States
(12) Patent Application Publication
(10) Pub. No.: US 2010/0094159 A1
(43) Pub. Date: Apr. 15, 2010

(54) APPARATUS FOR TRANSMITTING REFERENCE SIGNALS FOR MEASURING PHASE RESPONSE CHARACTERISTIC OF HUMAN BODY, AND PHASE RESPONSE CHARACTERISTIC MEASURING SYSTEM AND METHOD EMPLOYING THE SAME

(75) Inventors: Jung-Hwan Hwang, Daejon (KR); Sung-Weon Kang, Daejon (KR); Kyung-Soo Kim, Daejon (KR); Jung-Bum Kim, Daejon (KR); In-Gi Lim, Daejon (KR); Chang-Hee Hyoung, Daejon (KR); Jin-Bong Sung, Daejon (KR); Sung-Eun Kim, Seoul (KR); Duck-Gun Park, Daejon (KR); Jin-Kyung Kim, Daejon (KR); Hyung-Il Park, Daejon (KR)

Correspondence Address: AMPACC Law Group, 3500 188th Street S.W., Suite 103, Lynnwood, WA, 98037, US

(73) Assignee: Electronics and Telecommunications Research Institute, Daejon (KR)

(21) Appl. No.: 12/516,562

(22) PCT Filed: Oct. 29, 2007
(86) PCT No.: PCT/KR2007/005348

§ 371 Date: May 27, 2009

(30) Foreign Application Priority Data
Nov. 29, 2006 (KR) 1020060118984

(51) Int. Cl. A61B 5/053; H04B 5/00

(52) U.S. Cl. 600/547; 455/41.1

(57) ABSTRACT

Provided are an apparatus for transmitting reference signals for measuring the phase response characteristic of a human body in human body communication, and a system and method for measuring the phase response characteristic of a human body using the same. The apparatus including: a signal generator for generating reference signals for measuring the phase response characteristic of a human body; a signal distributor for distributing the generated reference signals to a transmitting electrode and a frequency converter; the transmitting electrode for applying the reference signals distributed by the signal distributor to a human body; the frequency converter for converting the frequency of the reference signals distributed by the signal distributor into a frequency different from a frequency of a reference signal applied to the human body via the transmitting electrode; and a radio antenna for wirelessly transmitting the frequency-converted reference signal to a free space.

### TECHNICAL FIELD

The present invention relates to an apparatus for transmitting reference signals for measuring the phase response characteristic of a human body in human body communication, and a system and method for measuring the phase response characteristic of a human body using the same; and more particularly, to an apparatus for transmitting reference signals for measuring the phase response characteristic of a human body, which can measure the phase response characteristic of a human body in human body communication in which no common ground line can be directly formed, and a phase response characteristic measuring system and method employing the same.

This work was supported by the IT R&D program for MIC/IITA. [2006-S072-01, “Controller SoC for Human Body Communications”.

### BACKGROUND ART

In modern societies, many people always carry various communication devices such as PDAs, cellular phones, and medical appliances. As methods of transmitting various data between these devices, there are (1) a wired transmission method using a cable and (2) a wireless transmission method using radio wave or light.

First, the wired transmission method is favorable in terms of security of data to be transmitted and transmission rate, but unfortunately, it is necessary to always carry a wired device, such as a cable.

In addition, the wireless transmission method is convenient to perform data transmission. However it needs additional circuits for wireless transmission and thus, it is difficult to provide at low costs.

To solve the above-mentioned problems, recently, a human body communication method using a human body as a transmission medium has been proposed. In other words, the human body communication method is a method in which a signal outputted through a transmitter of a communication device is applied to a human body via an electrode connected thereto and then transmitted through the human body, and the signal is received at a receiver of another communication device via another electrode connected to the human body. Such human body communication is convenient to use because no wired device such as a cable is needed, and it does not need additional circuits for wireless transmission.

In order to configure a communication system for this human body communication, it is necessary to analyze frequency characteristics of a human body corresponding to a channel of the communication system, and the phase response characteristic of a human body is required as one of the frequency characteristics.

FIG. 1 is an explanatory view of signal transfer characteristics in human body communication. In the drawing, a reference numeral “10” designates a signal transmitter, a reference numeral “11” represents a human body, a reference numeral “12” represents a signal receiver, a reference numeral “101” represents a signal transmitted through a human body, a reference numeral “102” indicates a signal transmitted by electromagnetic coupling, and a reference numeral “103” denotes an equivalent capacitor of an electromagnetic coupling component formed through the air between ground parts of the signal transmitter/receiver.

A signal outputted from the signal transmitter 10 is applied to the human body 11, and then transmitted through the human body 11 and inputted to the signal receiver 12.

Meanwhile, to achieve signal transmission, a closed circuit structure has to be established between the signal transmitter 10 and the signal receiver 12. In normal cases, this is accomplished by forming a common ground line between the ground parts of the signal transmitter 10 and the signal receiver 12.

However, in case of human body communication, it is impossible to form the common ground line. Instead, the ground part of the signal transmitter 10 and the ground part of the signal receiver 12 are electromagnetically coupled through the air, to thereby form a closed circuit structure between the signal transmitter 10 and the signal receiver 12.

FIG. 2 is an explanatory view of a method for measuring the phase response characteristic of a channel of a general communication system.

A phase measurement apparatus 23 measures the phase response characteristic of a channel by comparing the phase of a signal inputted to a general communication system channel 22 from a reference signal generator 21 and that of a signal outputted through the general communication system channel 22. At this time, the phases of an input signal and an output signal are measured with respect to a common ground line 24. In FIG. 2, reference numerals “25” and “26” represent the internal ground of the phase measurement apparatus.

The difference between the “output signal phase” and the “input signal phase” obtained by the phase measurement apparatus 23 becomes the “phase response characteristic of the general communication system”.

On the other hand, in human body communication, the ground part of the signal transmitter 10 and the ground contact part of the signal receiver 12 are not directly connected to each other but electromagnetically coupled through the air. Therefore, a common ground line cannot be formed between the signal transmitter 21 and the signal receiver 23, and thus, the phase response characteristic of a human body channel cannot be measured by the conventional phase response measurement method.

### DISCLOSURE OF INVENTION

### Technical Problem

It is, therefore, an object of the present invention to provide an apparatus for transmitting reference signals for measuring the phase response characteristic of a human body, which can measure the phase response characteristic of a human body in human body communication in which no common ground line can be directly formed, and a phase response characteristic measuring system and method employing the same.

In other words, it is an object of the present invention to provide an apparatus for transmitting reference signals for measuring the phase response characteristic of a human body in human body communication, which can measure the phase response characteristic of a human body while maintaining the condition of electromagnetic coupling in a structure where the grounds of a signal transmitter and a signal receiver are not directly connected to each other but electromagnetically coupled through the air, and a system and method for measuring the phase response characteristic of a human body using the same.

### Technical Solution

In accordance with an aspect of the present invention, there is provided an apparatus for transmitting reference signals for measuring the phase response characteristic of a human body in human body communication, the apparatus including: a signal generator for generating reference signals for measuring the phase response characteristic of a human body; a signal distributor for distributing the generated reference signals to a transmitting electrode and a frequency converter; the transmitting electrode for applying the reference signals distributed by the signal distributor to a human body; the frequency converter for converting the frequency of the reference signals distributed by the signal distributor into a frequency different from a frequency of a reference signal applied to the human body via the transmitting electrode; and a radio antenna for wirelessly transmitting the frequency-converted reference signal to a free space.

In accordance with another aspect of the present invention, there is provided a system for measuring the phase response characteristic of a human body in human body communication, the system including: a reference signal transmitter for applying to a human body some of reference signals for measuring the phase response characteristic of the human body and wirelessly transmitting the others of the reference signals to a free space; a reference signal receiver for receiving the reference signal transmitted via the free space; and a phase measurement apparatus for receiving the reference signal delivered through the human body and measuring the phase thereof, measuring the phase of the reference signal received at the reference signal receiver, and then calculating the phase response characteristic of the human body by comparison between the two measured phases.

In accordance with another aspect of the present invention, there is provided a method for measuring the phase response characteristic of a human body, the method including the steps of: generating reference signals for measuring the phase response characteristic of a human body; applying some of reference signals to a human body for the human body communication and wirelessly transmitting the others of the reference signals to a free space; receiving the reference signal transmitted via the free space and the reference signal delivered via the human body; and measuring the phase of each of the reference signals received at the step of receiving the reference signals; and calculating the phase response characteristic of the human body by comparison between the two phases measured at the step of measuring the phases.

### Advantageous Effects

As described above and will be set forth below, the present invention can measure the phase response characteristic of a human body (channel) in human body communication in which no common ground line can be formed.

In other words, the present invention can measure the phase response characteristic while maintaining the condition of electromagnetic coupling even in a structure where the grounds of a signal transmitter and a signal receiver are not directly connected, but electromagnetically coupled through the air.

Other objects and advantages of the present invention can be understood by the following description, and become apparent with reference to the embodiments of the present invention. Also, it is obvious to those skilled in the art to which the present invention pertains that the objects and advantages of the present invention can be realized by the means as claimed and combinations thereof.

### BRIEF DESCRIPTION OF THE DRAWINGS

FIG. 1 is an explanatory view of signal transfer characteristics in human body communication.

FIG. 2 is an explanatory view of a method for measuring the phase response characteristic of a channel of a general communication system.

FIG. 3 is an explanatory view of a system and method for measuring the phase response characteristic of a human body in human body communication in accordance with an embodiment of the present invention.

FIG. 4 illustrates a detailed configuration diagram of the reference signal transmitter shown in FIG. 3.

FIG. 5 illustrates a detailed configuration diagram of the reference signal receiver shown in FIG. 3.

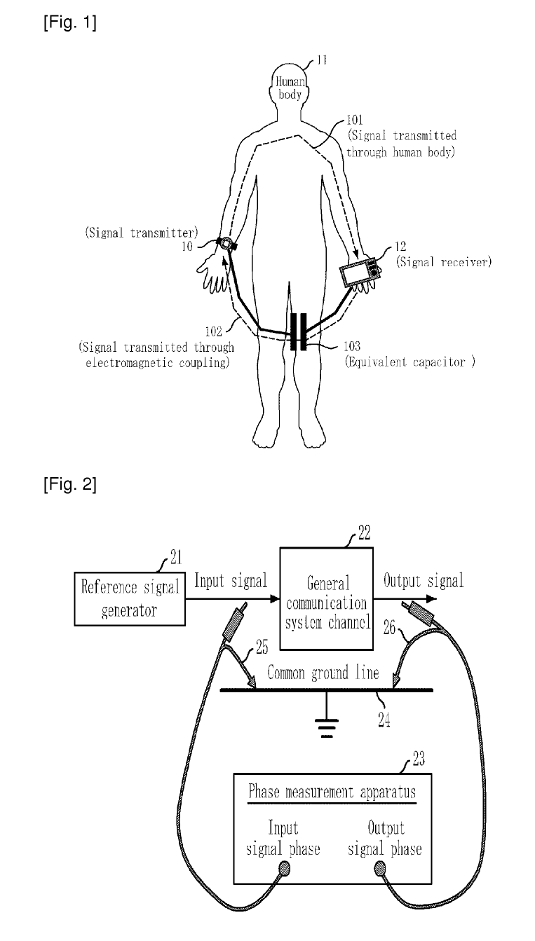

### BEST MODE FOR CARRYING OUT THE INVENTION

The advantages, features and aspects of the invention will become apparent from the following description of the embodiments with reference to the accompanying drawings, which is set forth hereinafter. By this, those skilled in the art will easily carry out the present invention. Further, in the following description, well-known arts will not be described in detail if it seems that they could obscure the invention in unnecessary detail. Hereinafter, preferred embodiments of the present invention will be set forth in detail with reference to the accompanying drawings.

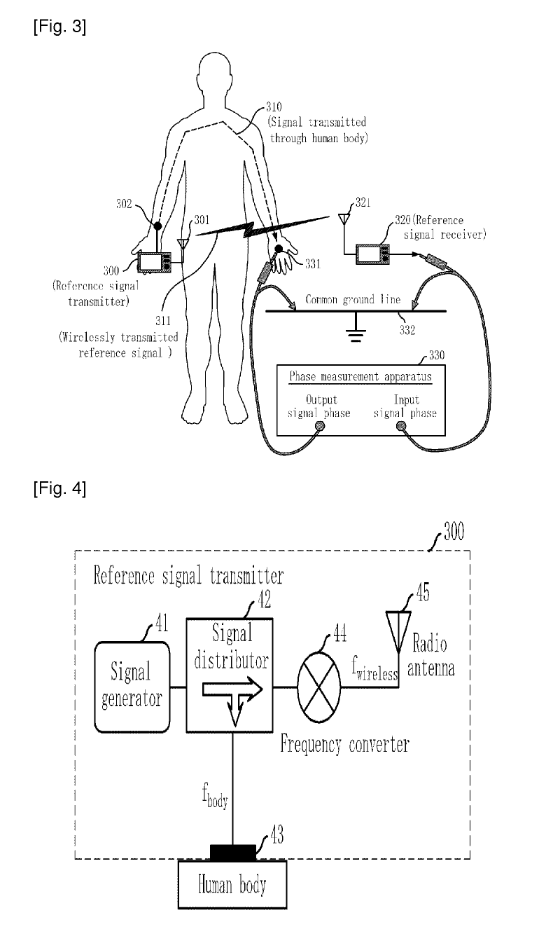

FIG. 3 is an explanatory view of a system and method for measuring the phase response characteristic of a human body in human body communication in accordance with an embodiment of the present invention.

The inventive system for measuring the phase response characteristic of a human body includes a reference signal transmitter 300, a reference signal receiver 320, and a phase measurement apparatus 330.

First, a measurement method of the system for measuring the phase response characteristic of a human body will be described below. In the present invention, some parts of reference signals generated by the reference signal transmitter 300 is transmitted through a human body to measure the phase of the signal, and at the same time, the other parts are wirelessly transmitted to measure its phase, and then the phases of the two signals are compared to acquire the phase characteristic of the human body. Here, the reference signal transmitted through the human body is received via a receiving electrode contacting the human body, and the reference signal received through a wireless space (free space) is received via the reference signal receiver 320 having a common ground line with the receiving electrode.

Hereinafter, each of the components will be described in detail.

The reference signal transmitter 300 outputs a reference signal via a radio antenna 301 and the transmitting electrode 302 contacting a human body.

Then, the reference signal receiver 320 receives the reference signal 311 wirelessly transmitted via the radio antenna 301 of the reference signal transmitter 300 by using a radio antenna 321.

Meanwhile, some of the reference signals generated by the reference signal transmitter 300 is applied to the human body via the transmitting electrode 302 contacting the human body, and then transmitted through the human body and reaches a receiving electrode 331. The receiving electrode 331 and the reference signal receiver 320 are connected by a common ground line.

The phase measurement apparatus 330 measures the phase of the reference signal received via the receiving electrode 331 and the phase of the reference signal received via the reference signal receiver 320, and then calculates the phase response characteristic of the human body by comparison between both phases.

The method for measuring the phase response characteristic of a human body channel will be described below in more detail.

The other reference signal except for the reference signal transmitted through a human body is transmitted in the air via the radio antenna 301 and received via the radio antenna 321 of the reference signal receiver, and then the phases of those signals are measured in the phase measurement apparatus 330. The reference signal transmitted through the air is transmitted at a very high speed, e.g., the speed of radio waves in a free space is 3×10⁸ m/s. Therefore, the signal received at the reference signal receiver 320 is almost the same as the signal transmitted at the reference signal transmitter 300.

Thus, the phase of the signal received via the reference signal receiver 320 is the same as that of the transmission signal of the reference signal transmitter 300, and compared with the phase of the signal 310 transmitted through the human body, thereby enabling the measurement of the phase response characteristic of the human body. At this time, as the phases of the two signals can be measured with respect to a common ground line 332, it is possible to measure the phase response characteristic of the human body by using a conventional phase response characteristic measurement method.

FIG. 4 illustrates a detailed configuration diagram of the reference signal transmitter depicted in FIG. 3.

The inventive reference signal transmitter 300 for measurement of the phase response characteristic of a human body includes a signal generator 41, a signal distributor 42, a transmitting electrode 43 corresponding to reference numeral “302” in FIG. 3, a frequency converter 44, i.e., a first frequency converter, and a radio antenna 45 corresponding to reference numeral “301” in FIG. 3. Hereinafter, each of the components will be described in detail.

The signal generator 41 generates reference signals. Then, the signal distributor 42 separates the signals to be transmitted to a human body via the transmitting electrode and to be wirelessly transmitted via the radio antenna.

The transmitting electrode 43 is in contact with the human body, and applies the reference signal separated as a signal for human body communication by the signal distributor 42 to the human body. The reference signal so applied is transmitted through the human body.

Meanwhile, the frequency converter 44 converts the reference signal separated as a signal for wireless transmission by the signal distributor 42 into a signal having a frequency f_{wireless} suitable for wireless transmission. The reference signal thus frequency-converted is then sent to a wireless space via the radio antenna 45.

In brief, some of the signals generated from the signal generator 41 is applied to a human body via the transmitting electrode 43, and the others of the reference signals is wirelessly transmitted via the radio antenna 45. At this time, the reference signal transmitted via the radio antenna 45 is frequency-converted by the frequency converter 44 so that it is not interfered with the signal applied to the human body and transmitted through the human body.

In other words, the frequency (f_{body} of FIG. 4) of the signal applied to the human body and transmitted through the human body and the frequency (f_{wireless} of FIG. 4) of the signal wirelessly transmitted are made different, thereby preventing mutual interference that may occur upon transmission of the two signals. At this time, a phase change that appears as the signal is converted by the frequency converter 44 can be detected by measurement of the characteristics of the frequency converter 44, and thus it can be compensated for when calculating the phase response characteristic.

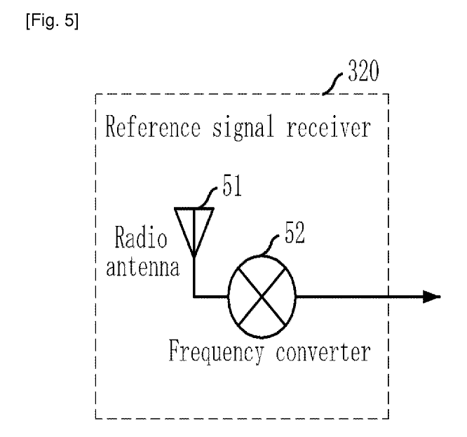

FIG. 5 is a detailed configuration diagram of the reference signal receiver depicted in FIG. 3.

As shown in the drawing, the reference signal receiver 320 includes a radio antenna 51 corresponding to reference numeral “321” of FIG. 3 and a frequency converter 52. Hereinafter, each of the components will be described in detail.

The radio antenna 51 receives a reference signal 311 that is wirelessly transmitted, and the frequency converter (second frequency converter) 52 frequency-converts the signal received via the radio antenna 51. That is, the reference signal transmitted through the radio antenna 301 of the reference signal transmitter 300 is wirelessly transmitted and received via the radio antenna 321 of the reference signal receiver 320.

As explained in FIG. 4, because the signal transmitted wirelessly so as to avoid interference with the signal transmitted through the human body is frequency-converted by the frequency converter 44 of the reference signal transmitter 300, the reference signal transmitter 320 converts this signal back into the original frequency signal by the frequency converter 52. At this time, a phase change that appears due to the signal conversion by the frequency converter 52 can be detected by measurement of the characteristics of the frequency converter 52, and thus it can be compensated for when calculating the phase response characteristic.

The method of the present invention as described above may be implemented by a software program that is stored in a computer-readable storage medium such as CD-ROM, RAM, ROM, floppy disk, hard disk, optical magnetic disk, or the like. This process may be readily carried out by those skilled in the art, and therefore, details of thereof are omitted here.

The present application contains subject matter related to Korean Patent Application No. 2006-0118984, filed in the Korean Intellectual Property Office on Nov. 29, 2006, the entire contents of which is incorporated herein by reference.

While the present invention has been described with respect to the particular embodiments, it will be apparent to those skilled in the art that various changes and modifications may be made without departing from the spirit and scope of the invention as defined in the following claims.

1. An apparatus for transmitting reference signals for measuring the phase response characteristic of a human body in human body communication, comprising:
a signal generator for generating reference signals for measuring the phase response characteristic of a human body;
a signal distributor for distributing the generated reference signals to a transmitting electrode and a frequency converter;
the transmitting electrode for applying the reference signals distributed by the signal distributor to a human body;
the frequency converter for converting the frequency of the reference signals distributed by the signal distributor into a frequency different from a frequency of a reference signal applied to the human body via the transmitting electrode; and
a radio antenna for wirelessly transmitting the frequency-converted reference signal to a free space.

2. A system for measuring a phase response characteristic of a human body in human body communication, comprising:
a reference signal transmitter for applying to a human body some of reference signals for measuring the phase response characteristic of the human body and wirelessly transmitting the others of the reference signals to a free space;
a reference signal receiver for receiving the reference signal transmitted via the free space; and
a phase measurement apparatus for receiving the reference signal delivered through the human body and measuring the phase thereof, measuring the phase of the reference signal received at the reference signal receiver, and then calculating the phase response characteristic of the human body by comparison between the two measured phases.

3. The system of claim 2, wherein the reference signal transmitter converts the frequency of the reference signal transmitted to the free space into a frequency different from the frequency of the reference signal applied to the human body to wirelessly transmit the converted signal.

4. The system of claim 3, wherein the reference signal transmitter includes:
a signal generator for generating the reference signals;
a signal distributor for distributing the generated reference signals to a transmitting electrode and a frequency converter;
the transmitting electrode for applying the reference signals distributed by the signal distributor to a human body;
a first frequency converter for converting the frequency of the reference signals distributed by the signal distributor; and
a radio antenna for wirelessly transmitting the frequency-converted signal to a free space.

5. The system of claim 2, wherein the reference signal receiver includes:
a radio antenna for receiving the reference signal transmitted via the free space; and
a second frequency converter for converting the frequency of the received reference signal into the original frequency.

6. The system of claim 2, wherein the phase measurement apparatus includes:
a receiving electrode for receiving the reference signal delivered through the human body,
where the receiving electrode and the reference signal receiver being connected by a common ground line.

7. The system of claim 6, wherein the phase measurement apparatus compensates for a phase change caused by frequency conversion at the first and the second frequency converters when calculating the phase response characteristic of the human body.

8. A method for measuring the phase response characteristic of a human body, comprising the steps of:
generating reference signals for measuring the phase response characteristic of a human body;
applying some of reference signals to a human body for the human body communication and wirelessly transmitting the others of the reference signals to a free space;
receiving the reference signal transmitted via the free space and the reference signal delivered via the human body;
measuring the phase of each of the reference signals received at the step of receiving the reference signals; and
calculating the phase response characteristic of the human body by comparison between the two phases measured at the step of measuring the phases.

9. The method of claim 8, wherein the step of applying and transmitting the reference signals converts the frequency of the reference signal transmitted to the free space into a frequency different from the frequency of the reference signal applied to the human body to wirelessly transmit the converted signal.

10. The method of claim 9, wherein the step of receiving the reference signals converts the frequency of the reference signal transmitted via the free space into the original frequency.

11. The method of claim 8, wherein the step of receiving the reference signals receives the two reference signals, in a state that a receiving electrode for receiving the reference signal delivered through the human body and a reference signal receiver for receiving the reference signal transmitted via the free space are connected by a common ground line.
